(19) United States
(12) Patent Application Publication
(10) Pub. No.: US 2025/0331735 A1
(43) Pub. Date: Oct. 30, 2025

(54) USE OF COMMERCIAL FLUOROPHORES IN ELECTROCHEMICAL THC DETECTION

(71) Applicant: Consumer Safety Technology, LLC, Des Moines, IA (US)

(72) Inventors: Evan Rashied Darzi, Phoenix, AZ (US); Randall Blake Hellman, Los Angeles, CA (US); Christina Rae Forbes, Mesa, AZ (US); Di Huang, Chandler, AZ (US)

(21) Appl. No.: 19/175,429

(22) Filed: Apr. 10, 2025

Related U.S. Application Data
(60) Provisional application No. 63/640,551, filed on Apr. 30, 2024.

(51) Int. Cl. A61B 5/08; A61B 5/00; A61B 5/097; G01N 27/327

(52) U.S. Cl. CPC A61B 5/082; A61B 5/097; A61B 5/4845; G01N 27/3275

(57) ABSTRACT

Embodiments herein relate to detection devices. In an embodiment, a method of detecting THC compounds, is included, the method includes receiving an exhaled breath sample; electrochemical processing of the exhaled breath sample using a fluorescent molecule to form a fluorescent-labeled THC adduct; purifying the fluorescent-labeled THC adduct; and determining an amount of THC in the exhaled breath sample based on a measured fluorescence of the purified fluorescent-labeled THC adduct; wherein the fluorescent-labeled THC adduct is formed without a highly-reactive handle. Other embodiments are also included herein.

This application claims the benefit of U.S. Provisional Application No. 63/640,551, filed Apr. 30, 2024, the content of which is incorporated herein by reference in its entirety.

### FIELD

Embodiments herein relate generally to detection devices, and more specifically to detection devices that utilize commercial fluorophores to detect THC compounds.

### BACKGROUND

Breath alcohol detection devices are used to measure an amount of alcohol in a user's breath. It is known that concentration of alcohol in a user's breath is closely proportional to the concentration of alcohol in the user's blood, which is typically the basis upon which intoxication is legally determined. Generally, a user blows into a mouthpiece of an alcohol detection device and a breath path is configured to transport at least a portion of the breath sample to a sensing element of the detection device. The capability to detect an amount of phenolic cannabinoid, such as tetrahydrocannabinol, in a user's breath, would be valuable for law enforcement, employers, and accountability partners. The concentration of phenolic cannabinoid in a user's breath typically correlates with recent use of cannabinoid products, such as marijuana.

### SUMMARY

In a first aspect, a method of detecting THC compounds, can be included. The method can include receiving an exhaled breath sample, electrochemical processing of the exhaled breath sample using a fluorescent molecule to form a fluorescent-labeled THC adduct, purifying the fluorescent-labeled THC adduct, and determining an amount of THC in the exhaled breath sample based on a measured fluorescence of the purified fluorescent-labeled THC adduct. The fluorescent-labeled THC adduct can be formed without a highly-reactive handle.

In a second aspect, in addition to one or more of the preceding or following aspects, or in the alternative to some aspects, the highly-reactive handle can include at least one of an azide, activated acid halides including an acid chloride, an acid fluoride, an acid bromide, and an acid iodide, an oxalyl halide, an activated ester including p-nitrobenzene, tetrafluorobenze, and an activated amide including an Weinreb amides (N,O-Dimethylhydroxyamine) and an acyl imidazolium.

In a third aspect, in addition to one or more of the preceding or following aspects, or in the alternative to some aspects, the fluorescent can include at least one of xanthene derivatives, a xanthene based fluorophore including rhodamine, fluorescein, fluorescein isothiocyanate (FITC), and Texas red, a cyanine fluorophore including Cy3 and Cy5, a phycobilin fluorophore including including phycoerythrin (PE), a BODIPY fluorophore including Fmoc-Trp-BODIPY, BODIPY 493/503, and 4′,6-diamidino-2-phenylindole (DAPI).

In a fourth aspect, in addition to one or more of the preceding or following aspects, or in the alternative to some aspects, the fluorescent can be rhodamine.

In a fifth aspect, in addition to one or more of the preceding or following aspects, or in the alternative to some aspects, the fluorescent-labeled THC adduct can be purified using chromatography.

In a sixth aspect, a method of detecting THC compounds, can be included. The method can include receiving an exhaled breath sample, electrochemical processing of the exhaled breath sample using a fluorescent molecule to form a fluorescent-labeled THC adduct, purifying the fluorescent-labeled THC adduct, and determining an amount of THC in the exhaled breath sample based on a measured fluorescence of the purified fluorescent-labeled THC adduct. The fluorescent-labeled THC adduct can be formed via an esterification reaction.

In a seventh aspect, in addition to one or more of the preceding or following aspects, or in the alternative to some aspects, the fluorescent can include at least one of xanthene derivatives, a xanthene based fluorophore including rhodamine, fluorescein, fluorescein isothiocyanate (FITC), and Texas red, a cyanine fluorophore including Cy3 and Cy5, a phycobilin fluorophore including including phycoerythrin (PE), a BODIPY fluorophore including Fmoc-Trp-BODIPY, BODIPY 493/503, and 4′,6-diamidino-2-phenylindole (DAPI).

In an eighth aspect, in addition to one or more of the preceding or following aspects, or in the alternative to some aspects, the fluorescent can be rhodamine.

In a ninth aspect, in addition to one or more of the preceding or following aspects, or in the alternative to some aspects, the fluorescent-labeled THC adduct can be purified using chromatography.

In a tenth aspect, a method of detecting THC compounds, can be included. The method can include receiving an exhaled breath sample, electrochemical processing of the exhaled breath sample using a dye to form a dye-labeled THC adduct, measuring a spectrum absorbance of the dye-labeled THC adduct, determining an amount of THC in the exhaled breath sample based on the measured spectrum absorbance of the dye-labeled THC adduct. The dye-labeled THC adduct can be formed without a highly-reactive handle.

In an eleventh aspect, in addition to one or more of the preceding or following aspects, or in the alternative to some aspects, the highly-reactive handle can include at least one of an azide, activated acid halides including an acid chloride, an acid fluoride, an acid bromide, and an acid iodide, an oxalyl halide, an activated ester including p-nitrobenzene, tetrafluorobenze, and an activated amide including an Weinreb amides (N,O-Dimethylhydroxyamine) and an acyl imidazolium.

In a twelfth aspect, a method of detecting THC compounds, can be included. The method can include receiving an exhaled breath sample, electrochemical processing the exhaled breath sample using a fluorescent or a dye to form a labeled THC adduct, wherein the labeled THC adduct can be labeled with at least one of the fluorescent and the dye, measuring a spectrum absorbance or a fluorescence of the labeled THC adduct, and determining an amount of

THC in the exhaled breath sample based on the measured photo physical properties of the labeled THC adduct. The labeled THC adduct can be formed without a highly-reactive handle.

In a thirteenth aspect, in addition to one or more of the preceding or following aspects, or in the alternative to some aspects, the highly-reactive handle can include at least one of an azide, activated acid halides including an acid chloride, an acid fluoride, an acid bromide, and an acid iodide, an oxalyl halide, an activated ester including p-nitrobenzene, tetrafluorobenze, and an activated amide including an Weinreb amides (N,O-Dimethylhydroxyamine) and an acyl imidazolium.

### BRIEF DESCRIPTION OF THE FIGURES

Aspects may be more completely understood in connection with the following figures (FIGS.), in which:

FIG. 1 is a schematic view of a detection device in accordance with various embodiments herein.

FIG. 2 is a reaction between THC and a fluorophore in accordance with various embodiments herein.

FIG. 3 is a reaction between THC and a fluorophore in accordance with various embodiments herein.

FIG. 4 is a method in accordance with various embodiments herein.

FIG. 5 is a method in accordance with various embodiments herein.

FIG. 6 is a computerized detection system in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Embodiments herein relate to intoxicant detection devices using commercial fluorophores to detect the intoxicants. In various embodiments, the intoxicants can be cannabis, such as tetrahydrocannabinol, present in a user's breath. In various embodiments, the intoxicants can be labeled with a fluorescent or dye using electrochemical processes to form a labeled THC adduct to allow for the amount of THC to be measured.

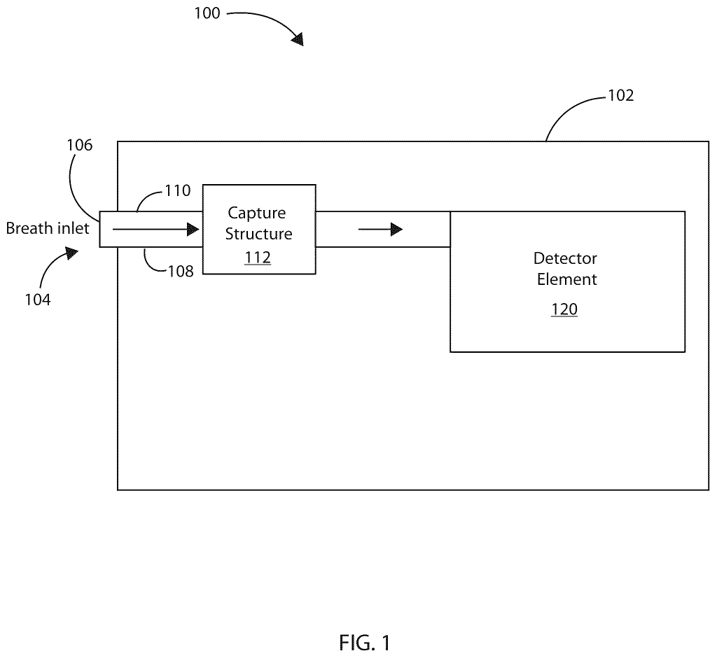

### Detection Device (FIG. 1)

Referring now to FIG. 1, a schematic view of a substance detection device is shown in accordance with various embodiments herein. In various examples, the detection device can detect a substance such as cannabis in a sample, such as a breath sample. The detection device 100 can include a housing 102 and a breath inlet 104. The housing 102 is preferably a relatively hard durable material that serves to protect the internal components of the detection device 100. The breath inlet 104 can be positioned on a side of the housing 102.

The detection device 100 can be used to measure amounts of intoxicants in a user's breath. For example, the detection device 100 can be used to measure the amount of phenolic cannabinoid, such as tetrahydrocannabinol, in the user's breath. The concentration of phenolic cannabinoid in a user's breath typically correlates with recent use of cannabinoid products, such as marijuana. Generally, a user blows into a mouthpiece of a detection device, and a breath path is configured to transport at least a portion of the breath sample to a capture structure of the detection device.

### Breath Opening/Mouthpiece

The breath inlet 104 can define a breath inflow opening 106. The breath inflow opening 106 can be configured to receive a user's breath. The breath inlet 104 can receive the mouth of the user providing a breath sample to the detection device 100. The breath inlet 104 can be configured to facilitate the user's mouth sealing against an exterior surface of the breath inlet 104. Alternatively, the breath inlet 104 can be configured to receive a breath sample that is provided where the user is spaced apart from the breath inlet 104 and is directing breath toward the breath inlet 104 from a distance.

In various embodiments, the breath inlet 104 can be configured to be removably attachable to the detection device 100. In some embodiments, the breath inlet 104 can include a mouthpiece. The mouthpiece can be removable by means of a friction or snap fit, or similar mechanism. This permits each user to have a separate mouthpiece for sanitary reasons, it also permits easy cleaning or replacement of the mouthpiece. In various embodiments, the breath inlet 104 can be formed from a substantially rigid material configured to retain its shape when a breath sample is provided to the detection device 100. Alternatively, the breath inlet 104 can be formed from a compliant material configured to conform to a user's mouth when a breath sample is provided to the detection device 100. The breath inlet 104 can be made from any suitable material or materials including but not limited to plastics, rubbers, silicone, metals, or the like.

In various embodiments, the user's breath can travel into the breath inflow opening 106 and through a breath conduit path 108. The breath conduit path 108 can define a breath path 110. In some embodiments, the breath conduit path 108 is connected to a capture structure 112 discussed below. The user's breath can travel into the breath inflow opening 106, through the breath path 110, and into or through the capture structure 112. It is herein contemplated that the capture structure 112 can capture components of one or more breaths of the user. In various embodiments, the capture structure 112 can capture components of one, two, three, four, five, six, seven, eight, nine, or ten breaths. For example, the capture structure 112 can capture components of one, two, three, four, or five breaths of the user.

### Capture Structure and Electrochemical Processing

In various embodiments, the capture structure 112 can include a material designed to capture or trap components found in the sample, such as the user's breath.

In various embodiments, a first solvent or solvent mixture can be added to the capture structure 112 in order to elute any cannabinoid compounds from the capture structure 112 and form a first solution. A basic buffer and commercial fluorophore or dye solution, discussed in more detail below, can then be added to the first solution. In various embodiments, the commercial fluorophore or dye can tag any cannabinoid compounds present in the first solution to form a fluorescent-labeled or dye-labeled THC adduct second solution. It is herein contemplated that the commercial fluorophores or dyes can tag the cannabinoid compounds using one or more electrochemical processes. Electrochemical processes can include any electrolytic platform that allows for the coupling of cannabinoid compounds with fluorophores or dyes. For example, an electrolytic cell or fuel cell can be used as the platform for the reaction. These electrochemical methods can include the use of either bulk electrochemical cells or flow cells and may include controlled-potential or controlled-current approaches. These reactions can include those promoted by a catalyst, electrocatalyst, or other chemical mediators. Exemplary electrochemical processes are disclosed in US Publ. No. 2023/0384286, titled “Systems and Methods for Oxidizing Phenolic Cannabinoids with Fuel Cells,” published Nov. 20, 2023, and assigned to ElectraTect, Inc., the content of which is hereby incorporated by reference in its entirety.

### Liquid Chromatography to Separate Cannabinoid Compounds

Once the fluorescent-labeled or dye-labeled THC adduct is formed, the nonpolar labeled THC adduct can be separated and thereby purified from any polar components that may be present in the second solution. A second solvent or solvent mixture can be added to the second solution and mixed thoroughly to form a third solution. After mixing, the third solution can be allowed to separate into polar and nonpolar phase layers, with the labeled THC adduct being contained with the nonpolar layer. Thereafter, the layers can be separated by solvent extraction. Exemplary liquid-based separation methods are disclosed in U.S. Pat. No. 11,026,596, titled “Detection and Measurement of Target Substance in Exhaled Breath,” patented on Jun. 8, 2011, and assigned to Hound Labs, Inc. the content of which is hereby incorporated by reference in its entirety. Further exemplary liquid chromatography separation methods are disclosed in U.S. Pat. No. 10,408,850, titled “Method for Target Substance Detection and Measurement,” patent on Sep. 10, 2019, and assigned to Hounds Labs, Inc. the content of which is hereby incorporated by reference in its entirety. It is noted that the Hound Labs references cited above require the use of highly-reactive handles to form a THC labeled adduct. These highly-reactive handles are described in more detail below. However, these highly-reactive handles are disadvantageous and embodiments described herein do not require these handles to form THC labeled adducts.

In other embodiments, it is contemplated herein that the fluorescent-labeled or dye-labeled THC adduct can be formed without the need to separate the components via liquid chromatography.

### Measuring the Amount of Cannabinoid Compounds in a Breath Sample

In various embodiments, the labeled THC adduct can travel to detector element 120. While FIG. 1 depicts the detector element 120 inside the housing 102, it is noted that the detector element 120 can be external to the housing 102. The detector element 120 can include a detector configured to measure the amount of cannabinoid compounds in the user's breath. The amount of cannabinoid compounds can be determined by measuring a photophysical property such as a spectrum absorbance or a fluorescence of the labeled THC adduct.

If the labeled THC adduct is tagged with a commercial fluorophore, the detector element 120 can include a light source configured to expose the fluorescent-labeled THC adduct to light and excite the fluorophore. The detector element 120 can include a variety of optical instruments configured to detect the amount of fluorescence. Optical instruments can include fluorescence microscopes, spectrofluorometers, fluorescence imaging systems, fluorescence lifetime imaging microscopes (FLIM), and the like. It is noted herein that the amount of fluorescence is directly correlated to the amount of cannabinoid compounds present in the user's breath.

Alternatively, if the labeled THC adduct is tagged with a commercial dye, the detector element 120 can include a light source configured to expose the dye-labeled THC adduct to light and the detector element 120 can include an instrument to measure the amount of light absorbed by the dye. Instruments can include spectrophotometers, UV-visible-near infrared (UV-Vis-NIR) spectrometers, photodiode array (PDA) detectors, and the like. It is noted herein that the amount of light absorbance is directly correlated to the amount of cannabinoid compounds present in the user's breath.

### Cannabinoid Compounds

Components of a sample can include compounds of interest which the detector element is designed to detect, such as cannabis. It is herein contemplated that cannabis, including a variety of cannabis metabolites or compounds, can be compounds of interest. Cannabis metabolites and cannabis compounds can include, but are not limited to, cannabinoids, phenolic cannabinoids, Δ⁹-tetrahydrocannabinol (Δ⁹-THC), Δ⁸-tetrahydrocannabinol (Δ⁸-THC), cannabinol (CBN), cannabidiol (CBD), 11-hydroxy-Δ⁹-THC (11-OH-THC), anandamide (arachidonylethanolamide), cannabichromene, and (−)Δ⁸-THC-11-oic acid).

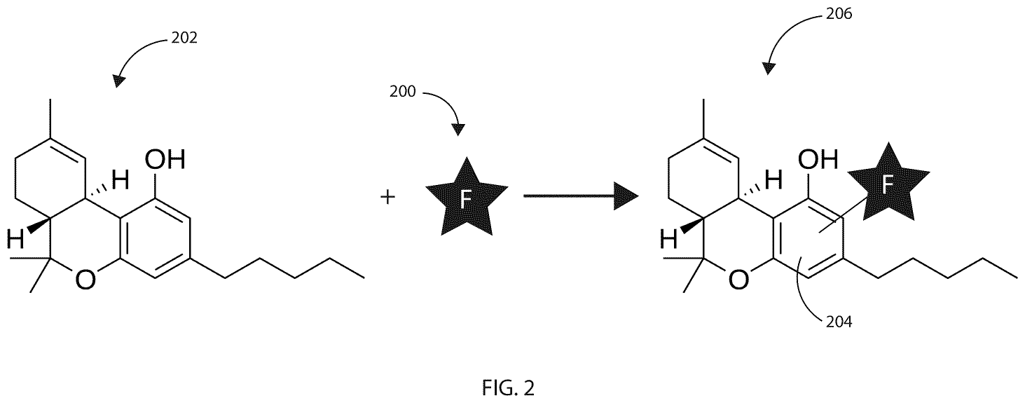

### Labeled-THC Adducts Formed Without Highly-Reactive Handles (FIG. 2)

In various embodiments herein, compounds of interest, such as THC, can be labeled or tagged with either commercials fluorophores or dyes using electrochemical processes. Notably, the electrochemical processes do not require the use of highly-reactive handles. Referring now to FIG. 2, a reaction between THC and a fluorophore is shown in accordance with various embodiments herein. In various embodiments, fluorophore 200 can selectively bind to THC molecule 202 at the para position and/or ortho position of the phenol ring 204. When the fluorophore 200 binds to the THC molecule 202, fluorophore-labeled THC adduct 206 is formed. The fluorophore 200 does not have a highly-reactive handle.

Highly-reactive handles can include any handle, coupled to a fluorophore or dye, that readily or vigorously undergoes a chemical reaction. For example, highly-reactive handles can readily undergo a chemical reaction with certain components in a breath sample or in the environment of the testing device. For example, the highly-reactive reactive handles can undergo a chemical reaction when exposed to cannabinoid compounds in the breath sample. Highly-reactive handles can include, but are not limited to, an azide, an oxalyl halide, an activated ester including p-nitrobenzene, tetrafluorobenze, and an activated amide including an Weinreb amides (N,O-Dimethylhydroxyamine), an acyl imidazolium, and the like. Highly-reactive handles can further include activated acid halides such as an acid chloride, an acid fluoride, an acid bromide, an acid iodide, and the like.

Fluorophores having highly-reactive handles are often explosive, unhealthy for human exposure, more likely to cause unintended reactions, or combinations of these. These fluorophores can also be very expensive. Rhodamine with an azide is an example of a fluorophore having a highly-reactive handle in the form of an azide which is easily explosive, unhealthy for human exposure, and expensive. By using a fluorophore without a highly-reactive handle instead of Rhodamine with an azide, it is possible to increase safety to humans, decrease the risk of an explosion, and decrease costs. In various embodiments, the fluorophores herein do not include an azide. For example, rhodamine does not have an azide.

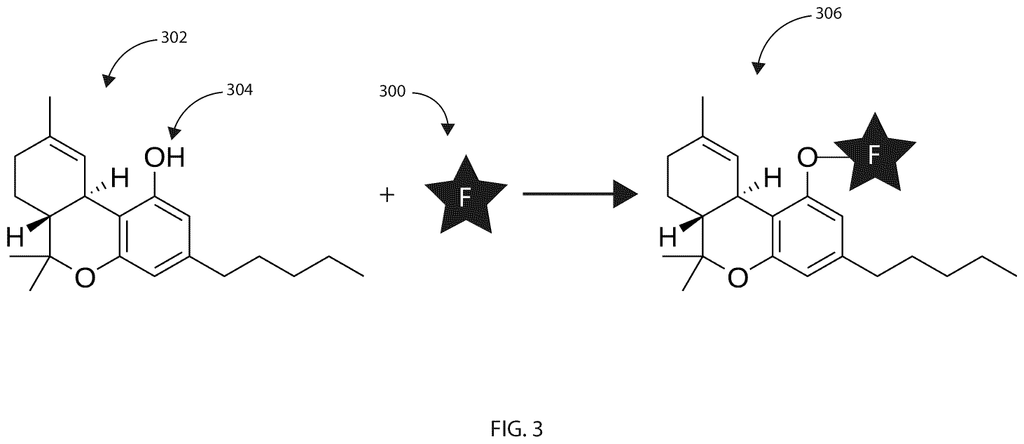

### Labeled-THC Adducts Formed Via Esterification Reaction (FIG. 3)

In various embodiments, the compounds of interest, such as THC, can be labeled or tagged with a commercial fluorophore or dye using an esterification reaction. Referring now to FIG. 3, a reaction between THC and a fluorophore is shown in accordance with various embodiments herein. In various embodiments, fluorophore 300 can selectively bind to THC molecule 302 at the hydroxyl group 304 of the phenol ring to form an ester bond. When the fluorophore 300 binds to the THC molecule 302, fluorophore-labeled THC adduct 306 is formed.

### Commercial Fluorophores

Commercial fluorophores can include any fluorophore capable of reacting with the compound of interest. Commercial fluorophores can include, but are not limited to, xanthene derivatives, a xanthene based xanthene-based fluorophore including rhodamine, tetramethylrhodamine, fluorescein, fluorescein isothiocyanate (FITC), Texas red, carboxyfluorescein (FAM), fluorescein-5-isothiocyanate, hexachlorofluorescein, and the like. Commercial fluorophores can further include a cyanine fluorophore including Cy2, Cy3, Cy5, and Cy7, a phycobilin fluorophore including phycoerythrin (PE), a BODIPY fluorophore including Fmoc-Trp-BODIPY, BODIPY 493/503, and 4′,6-diamidino-2-phenylindole (DAPI), DyLight® Fluor (available from Thermo Fisher Scientific, Waltham, MA 02451) and derivatives thereof, EverFluor (available from Setareh Biotech, LLC, Eugene OR 97402) and derivatives thereof.

Other commercial fluorophores can include cyanines, naphthalenes, coumarins, oxadiazoles, anthracenes, pyrenes, oxazines, acridines, arylmethines, tetrapyrroles, green fluorescent proteins, red fluorescent proteins, yellow fluorescent proteins, cadmium selenide quantum dots, cadmium selenide/zinc sulfide alloy quantum dots, cadmium selenide sulfide quantum dots, cadmium selenide sulfide/zine sulfide alloy quantum dots, cadmium telluride quantum dots, cadmium sulfide quantum dots, lead sulfide quantum dots, or indium phosphide/zinc sulfide alloy quantum dots, and derivatives thereof

### Commercial Dyes

Commercial dyes can include any dyes capable of reacting with the compound of interest. Commercial dyes can include, but are not limited to, cyanine dyes, alexa fluor dyes, and BODIPY dyes. Cyanine dyes can include Cy3, Cy5, and Cy7. Commercial dyes can further include, but are not limited to phenolphthalein, methyl orange, bromothylmol blue, methyl red, and Eriochrome Black T.

### Sample and Compounds of Interest

Throughout the application, breath is described as a sample that is analyzed for the presence of a substance such as an intoxicant. It is also possible for the embodiments of the application to be used to process a sample different than breath, such as another gas sample, such as environmental or ambient air or vapor from skin, or another biological sample, such as saliva, mucous, or urine. Breath and environmental or ambient air or vapor from skin provide the benefit of being noninvasive sample collection techniques to the user.

Throughout the application, cannabis is described as the substance of interest or compound of interest that is detected by a detector element. It is also possible for other substances and compounds to be detected by a detector element in the various embodiments described here in, such as different intoxicants, alcohol, prescription drugs, cocaine, heroin, nicotine, methamphetamine, amphetamines, hallucinogens, or other substances.

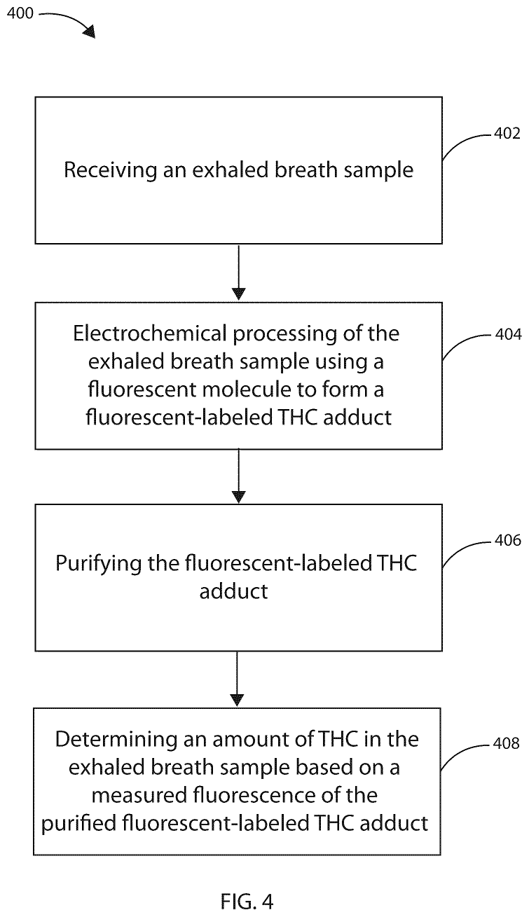

### Fluorescence Method (FIG. 4)

Many different methods for electrochemical THC detection using a fluorescent molecule are contemplated herein, including, but not limited to, methods of making, methods of using, and the like. Aspects of system/device operation described elsewhere herein can be performed as operations of one or more methods in accordance with various embodiments herein.

Referring now to FIG. 4, a flow diagram of a method is shown in accordance with various embodiments herein. FIG. 4 shows a method 400 of detecting THC compounds. The method can include receiving an exhaled breath sample 402.

The method can further include electrochemical processing of the exhaled breath sample using a fluorescent molecule to form a fluorescent-labeled THC adduct 404. In various embodiments, the electrochemical processing can include any electrolytic platform that allows for the coupling of cannabinoid compounds with fluorophores. In various embodiments, the fluorescent-labeled THC adduct is formed without the use of a highly-reactive handle. In various embodiments, the fluorescent-labeled THC adduct is formed using an esterification reaction.

The method can further include purifying the fluorescent-labeled THC adduct 406. In various embodiments, the fluorescent-labeled THC adduct is purified via liquid chromatography.

The method can further include determining an amount of THC in the exhaled breath sample based on a measured fluorescence of the purified fluorescent-labeled THC adduct 408. In various embodiments, the amount of THC is directly correlated to the measured fluorescence of the fluorescent-labeled THC adduct.

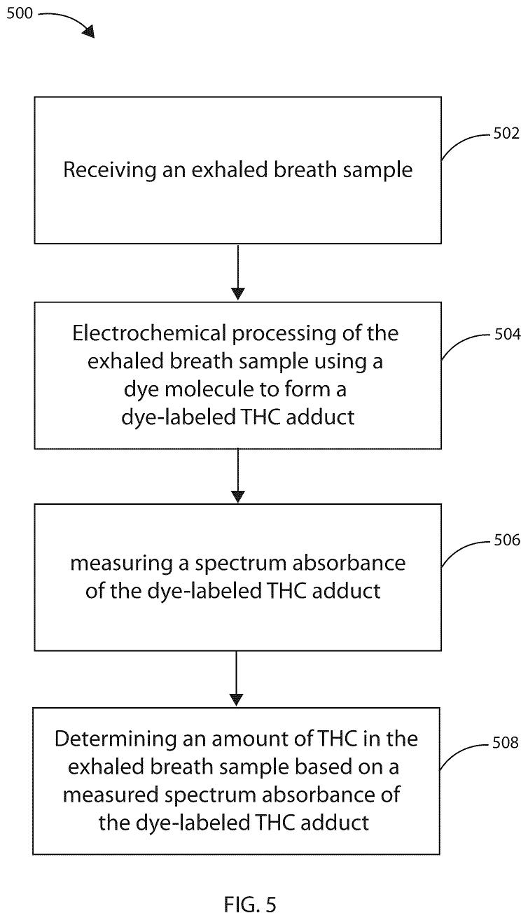

### Dye Method (FIG. 5)

Many different methods for electrochemical THC detection using a dye molecule are contemplated herein, including, but not limited to, methods of making, methods of using, and the like. Aspects of system/device operation described elsewhere herein can be performed as operations of one or more methods in accordance with various embodiments herein.

Referring now to FIG. 5, a flow diagram of a method is shown in accordance with various embodiments herein. FIG. 5 shows a method 500 of detecting THC compounds. The method can include receiving an exhaled breath sample 502.

The method can further include electrochemical processing of the exhaled breath sample using a dye molecule to form a dye-labeled THC adduct 504. In various embodiments, the electrochemical processing can include any electrolytic platform that allows for the coupling of cannabinoid compounds with dyes. In various embodiments, the dye-labeled THC adduct is formed without the use of a highly-reactive handle. In various embodiments, the dye-labeled THC adduct is formed using an esterification reaction.

The method can further include measuring a spectrum absorbance of the dye-labeled THC adduct 506.

The method can further include determining an amount of THC in the exhaled breath sample based on a measured spectrum absorbance of the dye-labeled THC adduct 508. In various embodiments, the amount of THC is directly correlated to the measured absorbance of the dye-labeled THC adduct.

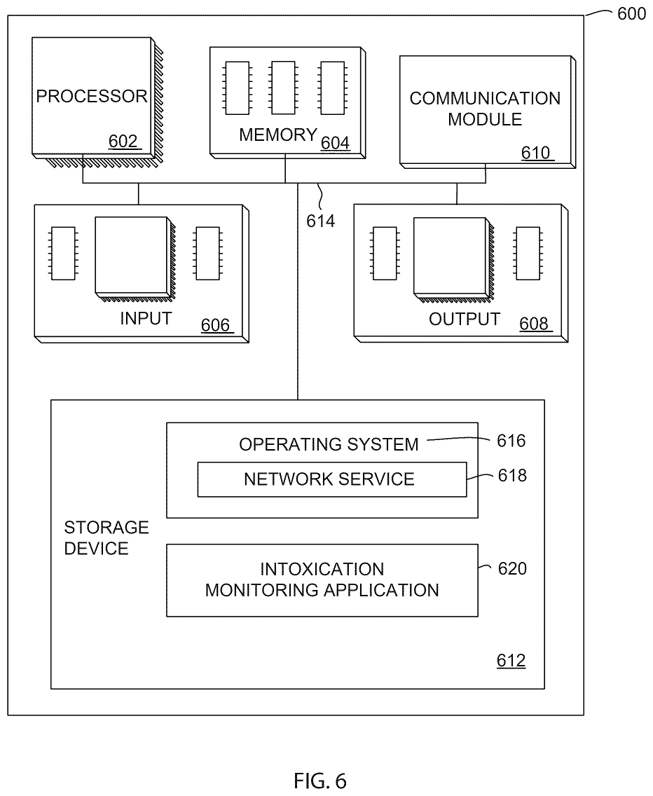

### Computer Systems (FIG. 6)

The systems and methods presented here may be implemented in part using a computerized device, such as a smartphone, handheld, or other computerized device. Computerized devices can be used to dispense solution or solvents, mix solutions, measure photo physical properties, display results, inform a user, and prompt action by a user, as well as for other functions. FIG. 6 shows a computerized detection system consistent with various examples described herein. FIG. 6 illustrates only one particular example of computing device 600, and other computing devices 600 may be used in other embodiments. Although computing device 600 is shown as a standalone computing device, computing device 600 may be any component or system that includes one or more processors or another suitable computing environment for executing software instructions in other examples and need not include all the elements shown here.

As shown in the specific example of FIG. 6, computing device 600 includes one or more processors 602, memory 604, one or more input devices 606, one or more output devices 608, one or more communication modules 610, and one or more storage devices 612. Computing device 600, in one example, further includes an operating system 616 executable by computing device 600. The operating system includes in various examples services such as a network service 618. One or more applications, such as a breath intoxicant detection application 620, are also stored on storage device 612 and are executable by computing device 600.

Each of components 602, 604, 606, 608, 610, and 612 may be interconnected (physically, communicatively, and/or operatively) for inter-component communications, such as via one or more communication channels 614. In some examples, communication channels 614 include a system bus, network connection, inter-processor communication network, or any other channel for communicating data. Applications such as breath intoxicant detection application 620 and operating system 616 may also communicate information with one another as well as with other components in computing device 600.

Processors 602, in one example, are configured to implement functionality and/or process instructions for execution within computing device 600. For example, processors 602 may be capable of processing instructions stored in storage device 612 or memory 604. Examples of processors 602 include any one or more of a microprocessor, a controller, a digital signal processor (DSP), an application specific integrated circuit (ASIC), a field-programmable gate array (FPGA), or similar discrete or integrated logic circuitry.

One or more storage devices 612 may be configured to store information within computing device 600 during operation. Storage device 612, in some examples, is known as a computer-readable storage medium. In some examples, storage device 612 comprises temporary memory, meaning that a primary purpose of storage device 612 is not long-term storage. Storage device 612 in some examples includes a volatile memory, meaning that storage device 612 does not maintain stored contents when computing device 600 is turned off. In other examples, data is loaded from storage device 612 into memory 604 during operation. Examples of volatile memories include random access memories (RAM), dynamic random-access memories (DRAM), static random-access memories (SRAM), and other forms of volatile memories known in the art. In some examples, storage device 612 is used to store program instructions for execution by processors 602. Storage device 612 and memory 604, in various examples, are used by software or applications running on computing device 600 such as breath intoxicant detection application 620 to temporarily store information during program execution.

Storage device 612, in some examples, includes one or more computer-readable storage media that may be configured to store larger amounts of information than volatile memory. Storage device 612 may further be configured for long-term storage of information. In some examples, storage devices 612 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

Computing device 600, in some examples, also includes one or more communication modules 610. Computing device 600 in one example uses communication module 610 to communicate with external devices via one or more networks, such as one or more wireless networks. Communication module 610 may be a network interface card, such as an Ethernet card, an optical transceiver, a radio frequency transceiver, or any other type of device that can send and/or receive information. Other examples of such network interfaces include Bluetooth, 3G, 4G, LTE, 5G, Wi-Fi radios, and Near-Field Communications (NFC), and Universal Serial Bus (USB). In some examples, computing device 600 uses communication module 610 to wirelessly communicate with an external device such as via public network such as the Internet. Computing device 600 also includes, in one example, one or more input devices 606. Input device 606, in some examples, is configured to receive input from a user through tactile, audio, or video input. Examples of input device 606 include a touchscreen display, a mouse, a keyboard, a voice responsive system, video camera, microphone, or any other type of device for detecting input from a user.

One or more output devices 608 may also be included in computing device 600. Output device 608, in some examples, is configured to provide output to a user using tactile, audio, or video stimuli. Output device 608, in one example, includes a display, a sound card, a video graphics adapter card, or any other type of device for converting a signal into an appropriate form understandable to humans or machines. Additional examples of output device 608 include a speaker, a light-emitting diode (LED) display, a liquid crystal display (LCD), or any other type of device that can generate output to a user.

Computing device 600 may include operating system 616. Operating system 616, in some examples, controls the operation of components of computing device 600, and provides an interface from various applications such as breath intoxicant detection application 620 to components of computing device 600. For example, operating system 616, in one example, facilitates the communication of various applications such as breath intoxicant detection application 620 with processors 602, communication unit 610, storage device 612, input device 606, and output device 608. Applications such as breath intoxicant detection application 620 may include program instructions and/or data that are executable by computing device 600. As one example, breath intoxicant detection application 620 may include instructions that cause computing device 600 to perform one or more of the operations and actions described in the examples presented herein. Instead of a breath intoxicant detection application 620, the system may include an intoxication interlock application, a personal monitoring application, a substance detection application, or other applications.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method of detecting THC compounds, comprising:
receiving an exhaled breath sample;
electrochemical processing of the exhaled breath sample using a fluorescent molecule to form a fluorescent-labeled THC adduct;
purifying the fluorescent-labeled THC adduct; and
determining an amount of THC in the exhaled breath sample based on a measured fluorescence of the purified fluorescent-labeled THC adduct;
wherein the fluorescent-labeled THC adduct is formed without a highly-reactive handle.

2. The method of claim 1, wherein the highly-reactive handle comprises at least one of an azide, activated acid halides including an acid chloride, an acid fluoride, an acid bromide, and an acid iodide, an oxalyl halide, an activated ester including p-nitrobenzene, tetrafluorobenze, and an activated amide including an Weinreb amides (N,O-Dimethylhydroxyamine) and an acyl imidazolium.

3. The method of claim 1, wherein the fluorescent comprises at least one of xanthene derivatives, a xanthene based fluorophore including rhodamine, fluorescein, fluorescein isothiocyanate (FITC), and Texas red, a cyanine fluorophore including Cy3 and Cy5, a phycobilin fluorophore including phycoerythrin (PE), a BODIPY fluorophore including Fmoc-Trp-BODIPY, BODIPY 493/503, and 4′,6-diamidino-2-phenylindole (DAPI).

4. The method of claim 3, wherein the fluorescent is rhodamine.

5. The method of claim 1, wherein the fluorescent-labeled THC adduct is purified using chromatography.

6. A method of detecting THC compounds, comprising:
receiving an exhaled breath sample;
electrochemical processing of the exhaled breath sample using a fluorescent molecule to form a fluorescent-labeled THC adduct;
purifying the fluorescent-labeled THC adduct; and
determining an amount of THC in the exhaled breath sample based on a measured fluorescence of the purified fluorescent-labeled THC adduct;
wherein the fluorescent-labeled THC adduct is formed via an esterification reaction.

7. The method of claim 6, wherein the fluorescent comprises at least one of xanthene derivatives, a xanthene based fluorophore including rhodamine, fluorescein, fluorescein isothiocyanate (FITC), and Texas red, a cyanine fluorophore including Cy3 and Cy5, a phycobilin fluorophore including including phycoerythrin (PE), a BODIPY fluorophore including Fmoc-Trp-BODIPY, BODIPY 493/503, and 4′, 6-diamidino-2-phenylindole (DAPI).

8. The method of claim 7, wherein the fluorescent is rhodamine.

9. The method of claim 6, wherein the fluorescent-labeled THC adduct is purified using chromatography.

10. A method of detecting THC compounds, comprising:
receiving an exhaled breath sample;
electrochemical processing of the exhaled breath sample using a dye to form a dye-labeled THC adduct;
measuring a spectrum absorbance of the dye-labeled THC adduct;
determining an amount of THC in the exhaled breath sample based on the measured spectrum absorbance of the dye-labeled THC adduct;
wherein the dye-labeled THC adduct is formed without a highly-reactive handle.

11. The method of claim 10, wherein the highly-reactive handle comprises at least one of an azide, activated acid halides including an acid chloride, an acid fluoride, an acid bromide, and an acid iodide, an oxalyl halide, an activated ester including p-nitrobenzene, tetrafluorobenze, and an activated amide including an Weinreb amides (N,O-Dimethylhydroxyamine) and an acyl imidazolium.

12. A method of detecting THC compounds, comprising:
receiving an exhaled breath sample;
electrochemical processing the exhaled breath sample using a fluorescent or a dye to form a labeled THC adduct, wherein the labeled THC adduct is labeled with at least one of the fluorescent and the dye;
measuring a spectrum absorbance or a fluorescence of the labeled THC adduct; and
determining an amount of THC in the exhaled breath sample based on the measured photo physical properties of the labeled THC adduct;
wherein the labeled THC adduct is formed without a highly-reactive handle.

13. The method of claim 12, wherein the highly-reactive handle comprises at least one of an azide, activated acid halides including an acid chloride, an acid fluoride, an acid bromide, and an acid iodide, an oxalyl halide, an activated ester including p-nitrobenzene, tetrafluorobenze, and an activated amide including an Weinreb amides (N,O-Dimethylhydroxyamine) and an acyl imidazolium.
